(19) United States
(12) Patent Application Publication
(10) Pub. No.: US 2020/0115301 A1
(43) Pub. Date: Apr. 16, 2020

(54) PROCESS FOR RECOVERY OF ETHYLENE FROM DRY GAS

(71) Applicant: UOP LLC, Des Plaines, IL (US)

(72) Inventors: David A. Roman, Guildford (GB); Joris Franken, Antwerp (BE); David Evans, Guildford (GB)

(21) Appl. No.: 16/600,707

(22) Filed: Oct. 14, 2019

Related U.S. Application Data
(60) Provisional application No. 62/745,867, filed on Oct. 15, 2018.

(51) Int. Cl. C07C 7/00; C07C 5/09; C07C 7/11; C07C 7/12

(52) U.S. Cl. CPC C07C 7/005; C07C 7/12; C07C 7/11; C07C 5/09

(57) ABSTRACT

A process for recovering ethylene from an FCC absorber off-gas stream comprising ethylene, ethane and heavier hydrocarbons and light gases involves removing hydrogen, nitrogen, sulfur species, carbon monoxide/dioxide, methane and other impurities from the off-gas. An absorption zone is upstream of an acetylene selective hydrotreating reactor to remove acid gases. An adsorption zone is downstream of the selective hydrotreating reactor to remove impurities that can impair ethylene recovery.

### CROSS-REFERENCE TO RELATED APPLICATIONS

This application claims priority from provisional application 62/745,867, filed Oct. 15, 2018, incorporated herein in its entirety.

### FIELD

The field relates to processes and apparatuses for recovery of ethylene from dry gas. More particularly, the technical field relates to processes and apparatuses for recovery of ethylene from FCC absorber off-gas.

### BACKGROUND

In a typical fluid catalytic cracking (FCC) unit, the absorber off-gas, also known as dry gas, contributes to approximately one-third of the refinery fuel gas production. Dry gas is the common name for the absorber off-gas stream that contains all the gases with boiling points lower than ethane. A typical dry gas stream contains 5 to 50 wt % ethylene, 10 to 20 wt % ethane, 5 to 20 wt % hydrogen, 5 to 20 wt % nitrogen, 0.05 to 5.0 wt % of carbon monoxide, 0.1 to 5.0 wt % of carbon dioxide and less than 0.01 wt % hydrogen sulfide and ammonia with the balance being methane and other impurities.

Valuable components are resident in the dry gas. Ethane can be good feed source for an ethane cracking facility for ethylene production and ethylene can be recovered for polyethylene production. Currently most ethylene and ethane in the dry gas is burned instead of recovered because the off-gas contains so many contaminants that are uneconomical to remove. However, dry gas streams still contain attractive quantities of ethylene and ethane if recovery could be made economical.

An FCC unit that processes 7,949 kiloliters (50,000 barrels) per day will generate and burn as much as 181,000 kg (200 tons) of dry gas containing about 36,000 kg (40 tons) of ethylene and 14,400 kg (16 tons) of ethane as fuel per day. Because a large price differential exists between fuel gas and pure ethylene or steam cracker feed it would be economically advantageous to recover this ethylene and ethane from dry gas.

Accordingly, it is desirable to provide apparatuses and processes for the removal of impurities from dry gas to allow recovery and use of ethylene in a safe and a cost-effective manner.

### BRIEF SUMMARY

A process for recovering ethylene from FCC absorber off-gas involves removing impurities from the off-gas. An absorption zone is upstream of a selective acetylene hydrotreating reactor to remove acid gases but an additional adsorption zone is downstream of the selective hydrotreating reactor to remove additional impurities.

### BRIEF DESCRIPTION OF THE DRAWING

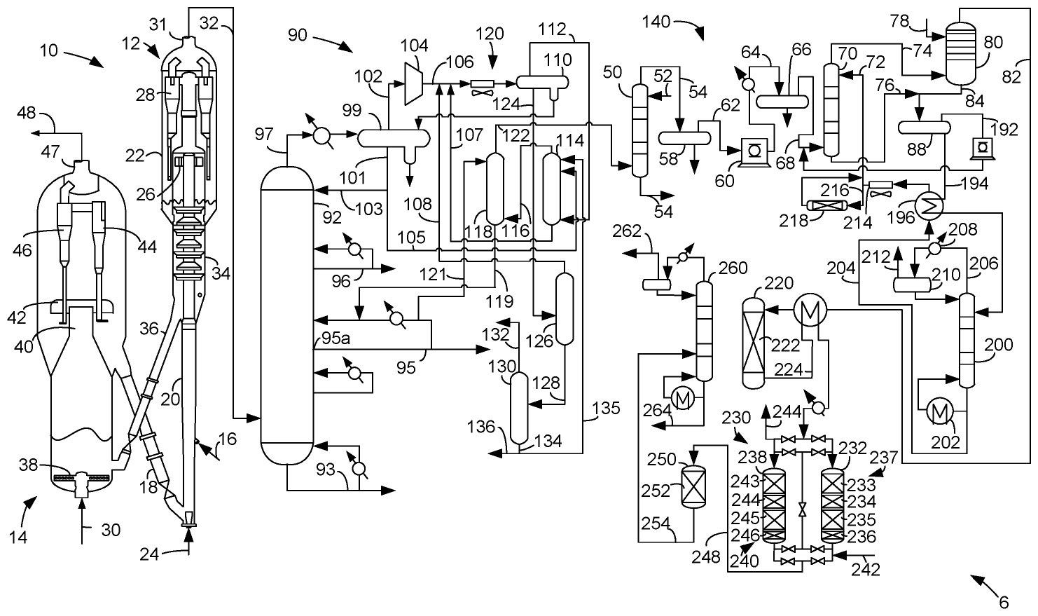

The FIG. 1s a schematic diagram of a process and an apparatus for recovery of a steam cracker feed in accordance with an exemplary embodiment.

### Definitions

As used herein, the term “stream” can include various hydrocarbon molecules and other substances.

The notation “C_{X}” means hydrocarbon molecules that have “x” number of carbon atoms, C_{X}⁺ means hydrocarbon molecules that have “x” and/or more than “x” number of carbon atoms, and C_{X}⁻ means hydrocarbon molecules that have “x” and/or less than “x” number of carbon atoms.

As used herein, the term “zone” can refer to an area including one or more equipment items and/or one or more sub-zones. Equipment items can include one or more reactors or reactor vessels, heaters, exchangers, pipes, pumps, compressors, controllers and columns. Additionally, an equipment item, such as a reactor, dryer, or vessel, can further include one or more zones or sub-zones.

As used herein, the term “overhead line” can mean a line connected at or near a top of a vessel, such as a column.

As used herein, the term “bottom stream” can mean a line connected at or near a bottom of a vessel, such as a column.

As depicted, process flow lines in the FIGURE can be referred to interchangeably as, e.g., lines, pipes, feeds, gases, products, discharges, parts, portions, or streams.

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take stripped product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

The term “predominant” means a majority, suitably at least 80 wt % and preferably at least 90 wt %.

### DETAILED DESCRIPTION

The present invention may be applied to any hydrocarbon stream containing ethylene, preferably, a dilute proportion of ethylene. A suitable, dilute ethylene stream may typically comprise between about 5 and about 50 wt % ethylene. An FCC dry gas stream is a suitable dilute ethylene stream. Other dilute ethylene streams may also be utilized such as a coker dry gas stream and other refinery off-gas streams. Because the present invention is particularly suited to FCC dry gas, the subject application will be described with respect to recovering ethylene from an FCC dry gas stream.

Now turning to the FIGURE, wherein like numerals designate like components, the FIGURE illustrates a refinery complex 6 that generally includes an FCC unit 10, a product recovery section 90 and a dry gas processing section 140. The FCC unit section 10 includes a reactor 12 and a catalyst regenerator 14. Process variables in the FCC unit 10 typically include a cracking reaction temperature of about 400° C. (752° F.) to about 600° C. (1112° F.) and a catalyst regeneration temperature of about 500° C. (932° F.) to about 900° C. (1652° F.). Both the cracking and regeneration occur at an absolute pressure below 506 kPa (72.5 psia).

The FIGURE shows a typical FCC reactor 12 in which a heavy hydrocarbon feed or raw oil stream in a distributor 16 is contacted with a regenerated cracking catalyst entering from a regenerated catalyst standpipe 18. Contacting in the FCC reactor 12 may occur in a narrow riser 20, extending upwardly to the bottom of a reactor vessel 22. The contacting of feed and catalyst is fluidized by gas from a fluidizing line 24. In an embodiment, heat from the catalyst vaporizes the hydrocarbon feed or oil, and the hydrocarbon feed is thereafter cracked to lighter molecular weight hydrocarbon products in the presence of the catalyst as both are transferred up the riser 20 into the reactor vessel 22. Inevitable side reactions occur in the riser 20 leaving coke deposits on the catalyst that lower catalyst activity. The cracked light hydrocarbon products are thereafter separated from the coked cracking catalyst using cyclonic separators which may include a primary separator 26 and one or two stages of cyclones 28 in the reactor vessel 22. Gaseous, cracked products exit the reactor vessel 22 through a product outlet 31 to line 32 for transport to a downstream product recovery section 90. The spent or coked catalyst requires regeneration for further use. Coked cracking catalyst, after separation from the gaseous product hydrocarbons, fall into a stripping section 34 where steam is injected through a nozzle to purge any residual hydrocarbon vapor. After the stripping operation, the coked catalyst is carried to the catalyst regenerator 14 through a spent catalyst standpipe 36.

The FIGURE depicts a regenerator 14 known as a combustor, although other types of regenerators are suitable. In the catalyst regenerator 14, a stream of oxygen-containing gas, such as air, is introduced through an air distributor 38 to contact the coked catalyst. Coke is combusted from the coked catalyst to provide regenerated catalyst and flue gas. The catalyst regeneration process adds a substantial amount of heat to the catalyst, providing energy to offset the endothermic cracking reactions occurring in the reactor riser 20. Catalyst and air flow upwardly together along a combustor riser 40 located within the catalyst regenerator 14 and, after regeneration, are initially separated by discharge through a disengager 42. Additional recovery of the regenerated catalyst and flue gas exiting the disengager 42 is achieved using first and second stage separator cyclones 44, 46, respectively within the catalyst regenerator 14.

Catalyst separated from flue gas dispenses through dip legs from cyclones 44, 46 while flue gas relatively lighter in catalyst sequentially exits cyclones 44, 46 and exits the regenerator vessel 14 through flue gas outlet 47 in flue gas line 48. Regenerated catalyst is carried back to the riser 20 through the regenerated catalyst standpipe 18. As a result of the coke burning, the flue gas vapors exiting at the top of the catalyst regenerator 14 in line 48 contain CO, CO₂, Na and H₂O, along with smaller amounts of other species. Hot flue gas exits the regenerator 14 through the flue gas outlet 47 in a line 48 for further processing.

The FCC product recovery section 90 is in downstream communication with the product outlet 31. In the product recovery section 90, the hot, gaseous FCC product in line 32 is directed to a lower section of an FCC main fractionation column 92. The main fractionation column 92 is also in downstream communication with the product outlet 31. Several fractions of FCC product may be separated and taken from the main fractionation column including a heavy slurry oil from the bottoms in line 93, a light cycle oil in line 95 taken from outlet 95a and a heavy naphtha stream in line 96 taken from outlet 96a. Any or all of streams in lines 93-96 may be cooled and pumped back to the main fractionation column 92 to cool the main fractionation column typically at a higher location. Gasoline and gaseous light hydrocarbons are removed in an overhead line 97 from the main fractionation column 92 and condensed before entering a main column receiver 99. The main column receiver 99 is in downstream communication with the product outlet 31.

An aqueous stream is removed from a boot in the main column receiver 99.

Moreover, a condensed light naphtha stream is removed in a condensate line 101 while an overhead stream is removed in an overhead line 102 from the receiver 99. The overhead stream in the overhead line 102 contains gaseous light hydrocarbons which may comprise a dilute ethylene stream. A portion of the condensed stream in condensate line 101 is refluxed back to the main column in line 103, so the main fractionation column 92 is in upstream communication with the main column receiver 99. A net liquid bottoms stream in a net bottoms line 105 and a net gaseous overhead stream in overhead line 102 comprising unstabilized light naphtha may enter a gas recovery section 120 of the product recovery section 90.

The gas recovery section 120 is shown to be an absorption based system, but any gas recovery system may be used, including a cold box system. To obtain sufficient separation of light gas components the gaseous stream in overhead line 102 is compressed in compressor 104. More than one compressor stage may be used, and typically a dual stage compression is utilized to compress the gaseous stream in line 102 to between about 1.2 MPa (gauge) (180 psig) to about 2.1 MPa (gauge) (300 psig) to provide a compressed light vaporous hydrocarbon stream. Three stages of compression may be advantageous to provide additional pressure at least as high as 3.4 MPa (gauge) (500 psig).

The compressed light vaporous hydrocarbon stream in a compressor discharge line 106 may be joined by streams in lines 107 and 108, cooled and delivered to a high pressure receiver 110. An aqueous stream from the receiver 110 may be routed to the main column receiver 99. A gaseous hydrocarbon stream in a high pressure overhead line 112 from the top of the high pressure receiver 110 is routed to a lower end of a primary absorber column 114. In the primary absorber column 114, the gaseous hydrocarbon stream is contacted with the unstabilized light naphtha stream from the net main column receiver bottoms stream in the net main column receiver bottoms line 105 directed to an upper end of the primary absorber column 114 to effect a separation between C₃₊ and C₂₋ hydrocarbons. This separation is further improved by feeding stabilized gasoline from line 135 above the feed inlet for stream 105. The primary absorber column 114 is in downstream communication with an overhead line 102 of the main column receiver via the compressor discharge line 106, the high pressure overhead line 112 and the main column bottoms line 105 of the main column receiver 99. A liquid C₃₊ bottoms stream in an absorber bottoms line 107 is returned to the compressor discharge line 106 prior to cooling. A primary off-gas stream in a primary absorber overhead line 116 from the primary absorber column 114 comprises a dilute ethylene stream which is fed to the lower end of a secondary absorber 118.

The secondary absorber column 118 is in downstream communication with the primary absorber column 114. A circulating stream of light cycle oil in line 121 diverted from line 95 to an upper end of the secondary absorber column 118 absorbs most of the C₃-C₄ material in the primary off-gas. The secondary absorber column 118 is in downstream communication with the main fractionation column 92. Light cycle oil from the bottom of the secondary absorber column 118 in the secondary absorber bottoms line 119 rich in C₃₊ material is returned to the main fractionation column 92 via the pump-around for line 95. The main fractionation column 92 is in downstream communication with the secondary absorber column 118 via the secondary absorber bottoms line 119. A secondary off-gas stream from the secondary absorber column 118 comprising dry gas of predominantly C₂₋ hydrocarbons with many impurities is removed in the secondary absorber overhead line 122 as a hydrocarbon stream to be further processed. Both of the absorber columns 114 and 118 have no condenser or reboiler, but may employ pump-around cooling circuits.

In the high pressure receiver 110, the gaseous hydrocarbon stream exiting in the high pressure overhead line 112 is separated from a high pressure liquid stream comprising C₃₊ hydrocarbons exiting from the bottom of the high pressure receiver 110 in a high pressure bottoms line 124. The high pressure liquid stream in the high pressure bottoms line 124 is sent to a stripper column 126. The stripper column 126 has no condenser but receives the cooled high pressure liquid stream in the high pressure bottoms line 124. Most of the C₂₋ material is removed in the stripper overhead line 108 from the stripper column 126 and is returned to the compressor discharge line 106. A liquid stripper bottoms stream from the stripper column 126 is sent to a debutanizer fractionation column 130 through the stripper bottoms line 128.

The debutanizer fractionation column 130 produces a debutanizer overhead stream in a debutanizer overhead line 132 comprising C₃-C₄ hydrocarbon product and a debutanized bottoms stream in a debutanized bottoms line 134 comprising stabilized gasoline. A portion of the stabilized gasoline in the debutanized bottoms line 134 may be recycled in a debutanized recycle line 135 to a top of the primary absorber column 114 above the feed inlet for the main column receiver bottoms line 105 to improve the absorptive recovery of C₃₊ hydrocarbons. The debutanizer overhead stream in the debutanizer overhead line 132 comprising C₃ and C₄ olefins may be used as feed for alkylation or subjected to further processing to recover olefins. In an aspect, the debutanizer overhead line 132 may be fed to a LPG splitter column to split C₃ hydrocarbons from C₄ hydrocarbons. A net debutanized bottom stream in a net debutanized bottoms line 136 may be fractionated in a naphtha splitter column to separate light and heavy naphtha and/or further treated and sent to gasoline storage.

Table 1 shows the range of impurities in the secondary off-gas stream in the secondary absorber overhead line 122 from the secondary absorber column 118 comprising a dry gas hydrocarbon stream and a typical maximum concentration required for ethylene recovery.

TABLE 1

| Contaminant | Dry Gas | Feed Maximum |
|---|---|---|
| H₂S, vppm | 5000-15000 | 0.25 |
| Mercaptans, vppm | 1-10 | 0.11 |
| COS, vppm | 5-20 | 0.01 |
| CO₂, vppm | 10000-20000 | 1 |
| CO, mol % | 0.1-5 | 0.77 |
| NOx, wppm | 10-500 | 1 |
| O₂, mol % | .05-5 | 0.0001 |
| Acetylene, vppm | 50-200 | 1 |
| NH₃, vppm | 0.1-5 | 0.1 |
| AsH₃, wppb | 50-500 | 15 |
| Mercury, wppb | 1-5 | 0.01 |
| HCN, vppm | 0.1-5 | 0.25 |
| CH₃OH, wppm | 0.1-5 | 1 |
| Water, vppm | saturated | 1 |

Table 2 shows the range of hydrocarbons and hydrogen that can be present in the dry gas hydrocarbon stream for recovery.

TABLE 2

| Component | mol % |
|---|---|
| Hydrogen | 5-30 |
| Nitrogen | 5-30 |
| Methane | 25-50 |
| Ethane | 10-20 |
| Ethylene | 10-30 |
| Propane | 0.25-1.5 |
| Propylene | 1-5 |
| Iso-Butane | 0.1-1.5 |
| n-Butane | 0.01-0.5 |
| 1-Butene | 0.05-0.75 |
| Iso-Butene | 0.05-0.75 |
| Trans-Butene | 0.05-0.75 |
| Cis-Butene | 0.05-0.75 |
| Butadiene | 0-0.1 |
| iso-Pentane | 0.05-0.5 |
| n-Pentane | 0-0.05 |
| C₅₊ | 0.05-0.5 |

The dry gas stream may have a temperature of about 25° C. (77° F.) to about 75° C. (167° F.) and a pressure of about 500 kPa (72 psig) to about 1500 kPa (217 psig). The hydrocarbon stream in the secondary overhead line 122 must be purified in the dry gas processing section 140 to allow for further processing to enable ethylene recovery. The dry gas processing section 140 may include an optional scrubber column 50, a compressor 60, an absorber column 70, a regenerator column 80, a selective hydrotreating reactor 220, a first adsorption unit 240, a second adsorption unit 250 and an ethylene splitter column 260.

The hydrocarbon stream in the secondary overhead line 122 may be fed to an up-front water wash column (not shown) to remove both chlorides and ammonia from the hydrocarbon stream. The hydrocarbon stream in the secondary overhead line 122 may be fed to a bottom of an optional scrubber column 50. In the scrubber column 50, carbon dioxide, hydrogen sulfide and carbonyl sulfide are absorbed from the hydrocarbon stream by counter-current contact with a scrubber solvent fed to the top of the scrubber column 50 in a scrubber solvent line 52. The hydrocarbon stream may be passed through the trayed or packed scrubber column 50 to provide a scrubbed hydrocarbon stream. The scrubber column 50 may be in downstream communication with the primary absorber column 114 and/or the secondary absorber column 118. Acid gases, hydrogen sulfide, carbon dioxide and carbonyl sulfide, are absorbed into the scrubber solvent from line 52. Preferred scrubber solvents include Selexol™ available from UOP LLC in Des Plaines, Ill. and amines such as alkanolamines including diethanol amine (DEA), monoethanol amine (MEA), methyl diethanol amine (MDEA), diisopropanol amine (DIPA), and diglycol amine (DGA). Other amines can be used in place of or in addition to the preferred amine solvents. The resultant scrubbed hydrocarbon stream exits an overhead of the scrubber column 50 in a scrubber overhead line 54 with about 5 to about 30 vppm of hydrogen sulfide still remaining in the scrubbed hydrocarbon stream. A hydrogen sulfide-rich solvent stream is taken out from a bottom of the scrubber column 50 in a scrubber bottoms line 54. The hydrogen sulfide-rich solvent from the bottom may be regenerated and recycled back to the scrubber column 50 in the scrubber solvent line 52. The scrubber column 50 may be operated at a temperature of about 40° C. (104° F.) to about 125° C. (257° F.) and a pressure of about 1200 to about 1600 kPa. The temperature of the scrubber solvent stream in the scrubber solvent line 52 may be between about 20° C. (68° F.) and about 70° C. (158° F.).

The optionally scrubbed hydrocarbon stream may be flashed in a scrubber knock-out drum 58 to remove liquid from the scrubbed hydrocarbon stream before it is transported to the compressor 60 in a scrubber knock-out overhead line 62. The compressor increases the pressure of the scrubbed hydrocarbon stream to a pressure of about 2000 kPa (g) (290 psig) to about 3000 kPa (g) (435 psig) perhaps with one or two reciprocating compressors.

The compressed hydrocarbon stream in a compressor discharge line 64 still requires further removal of acid gases from the compressed hydrocarbon stream. The compressed, hydrocarbon stream is cooled to about 20° C. (68° F.) to about 70° C. (158° F.) and flashed in a compressor knock out drum 66 to remove condensed components and fed to an absorber column 70 in a compressor knock-out overhead line 68. The compressed hydrocarbon stream in the compressor knock-out overhead line 68 may be fed to a bottom of the absorber column 70 and counter-currently contacted with an absorbent solvent fed to a top of the absorber column 70 in a lean solvent line 72. The compressed hydrocarbon stream may be passed through the trayed or packed absorber column 70. The absorber column 70 may be in downstream communication with the scrubber column 50 and the compressor 60. Acid gases, hydrogen sulfide, carbon dioxide and carbonyl sulfide, are absorbed into the absorbent solvent from the lean solvent line 72. Preferred absorbent solvents include Selexol and alkanolamines as previously mentioned for the scrubber solvent stream in the scrubber solvent line 52. The absorber column 70 may use an activator in the absorbent solvent that accelerates kinetics and reduces the number of required trays. The activator may comprise piperazine. Other amines can be used in place of or in addition to the preferred amines. The acid gas in the compressed hydrocarbon stream is absorbed from the gas phase into the liquid phase as the gas flows upwardly through the absorber column 70. The resultant absorbed, compressed hydrocarbon stream exits an overhead of the absorber column 70 in an absorber overhead line 74 with hydrogen sulfide, carbon dioxide and carbonyl sulfide concentrations reduced in the absorbed, compressed hydrocarbon stream to manageable levels. The absorbed, compressed hydrocarbon stream in the absorber overhead line 74 is transported to an absorber gas knock-out drum 80. An acid gas-rich absorbent solvent stream exits the absorber column in an absorber bottoms line 76 and is fed to the rich solvent flash drum 88.

The absorber gas knock-out drum 80 captures and separates solvent carryover in the absorbed, compressed hydrocarbon stream to reduce absorbent solvent losses. The top of the absorber gas knock-out drum 80 contains several water washing trays. The gaseous absorbed, compressed hydrocarbon stream flashes in the absorber gas knock-out drum 80 to separate from the solvent which exits in a drum bottoms line 84 and is fed with the acid gas-rich absorbent solvent in absorber bottoms line 76 to a rich solvent flash drum 88. Water fed from a water line 78 above the trays washes the solvent from the up-flowing gaseous absorbed, compressed hydrocarbon stream and exits the absorber gas knock-out drum 80 in an absorber gas drum overhead line 82. A hydrophilic mesh may be installed in the top of the absorber gas knock-out drum 80 to agglomerate solvent which tends to descend in the drum to avoid exit with the gaseous absorbed, compressed hydrocarbon stream in the absorber gas drum overhead line 82.

The rich solvent flash drum 88 is used to remove hydrocarbons that were co-absorbed with the acid gas. The vent gas from the rich solvent flash drum in the rich solvent flash overhead line 192, which has a relatively small flow rate, can be compressed and recycled back to the absorber column 70 via the compressor knock-out overhead line 68 or sent to an alternate destination. The rich solvent in the rich flash bottoms line 194 is sent to the lean/rich exchanger 196 where the temperature of the rich solvent in the rich flash bottoms line 194 is increased by heat exchange with the lean solvent in a regenerator bottoms line 204. The heated, rich solvent in the rich flash bottoms line 194 exiting the lean/rich exchanger 196 may be routed to the top of a regenerator column 200.

In the regenerator column 200, the heated rich solvent is thermally regenerated by rising steam vaporized in a regenerator reboiler 202. The regenerator reboiler 202 may use steam or hot oil for heating and partially vaporizes steam from the solvent in the regenerator reboiler. The rising steam strips acid gases from the rich solvent in the regenerator column 200. A regenerator overhead stream comprising acid gases and steam from the overhead of the regenerator 200 is partially condensed in a reflux condenser 208 and sent to the reflux drum 210. Condensate from the reflux drum 210 is returned to the regenerator column 200 as reflux and a net acid gas stream comprising carbon dioxide, carbonyl sulfide and hydrogen sulfide from a top of the reflux drum 210 is processed downstream in a net acid gas line 212. The lean solvent stream stripped of acid gases exits the bottom of the regenerator column in the regenerator bottoms line 204 and enters the lean/rich exchanger 196 to be cooled by heat exchange with the cooler rich solvent in the rich flash bottoms line 194. The regenerator column 200 may be operated at a bottoms temperature of about 100° C. (212° F.) to about 150° C. (302° F.), preferably no more than 136° C. (277° F.), and an overhead pressure of about 69 kPa (g) (10 psig) to about 207 kPa (g) (30 psig).

The cooled lean solvent stream in the regenerator bottoms line 204 from the lean/rich exchanger 196 may be sent to a lean solvent cooler 214 where it is cooled and returned to the absorber column 70 in the lean solvent line 72. A slip stream of cooled lean solvent in filter line 216 may be diverted to a filter 218 to remove solid impurities and returned to the lean solvent line 72 for recycle to the absorber column 70.

The compressed, absorbed hydrocarbon stream in the absorber gas drum overhead line 82 comprises acetylenes, nitrous oxides and oxygen which may be harmful to downstream processing. Hence, the compressed, absorbed hydrocarbon stream may be introduced to a selective hydrotreating reactor 220. The selective hydrotreating reactor 220 includes a selective hydrogenation catalyst in a fixed catalyst bed 222 for conversion of acetylene to ethylene. The selective hydrogenation catalyst minimizes full saturation of acetylene to ethane to retain valuable ethylene. No more than 10 wt %, suitably 5 wt % and preferably 1 wt % of ethylene in the compressed, absorbed hydrocarbon stream is converted to ethane. Moreover, the selective hydrogenation catalyst will preferably convert acetylene in the compressed, absorbed hydrocarbon stream to ethylene, as opposed to ethane, at a selectivity of at least about 60% and most preferably at least 80%. The selective hydrogenation catalyst also hydrogenates all nitrous oxides to ammonia and all oxygen to water which products are easier to remove from the compressed, absorbed hydrocarbon stream. The catalyst bed may use a nickel catalyst such as OleMax 102 available from Clariant Corporation in Louisville, Ky. Hydrogen originally resident in the compressed, absorbed hydrocarbon stream will be in sufficient quantity to selectively hydrogenate acetylene as well as to reduce both NOₓ and O₂ concentrations to trace levels. The hydrogen originally resident in the compressed, absorbed hydrocarbon stream was present in the original secondary off-gas stream in the secondary overhead line 122 and did not have to be added prior to entry into the selective hydrotreating reactor 220. Impurities in the compressed, absorbed hydrocarbon stream transported in the absorber gas drum overhead line 82 are not sufficient to impair operation of the selective hydrogenation at this point in the process.

The selective hydrotreating reactor should be run at about 200 to about 260° C., a pressure of between about 690 kPa (g) (100 psig) to about 4000 kPa (g) (580 psig) and may be run under adiabatic conditions. The hydrogenation catalyst should be presulfided such as with dimethyl disulfide to convert the hydrogenation metal from a nickel oxide to a nickel sulfide. The catalyst may be subjected to regeneration by coke removal using steam, air and an inert gas stream as carrier gas. The selective hydrotreating reactor 220 provides a hydrogenated, absorbed, compressed hydrocarbon stream in hydrogenation line 224.

After selective hydrogenation, several impurities remain in the hydrogenated, absorbed, compressed hydrocarbon stream and must be removed. The hydrogenated, absorbed, compressed hydrocarbon stream will still contain unacceptably high concentrations of one or more of mercury, carbon dioxide, carbonyl sulfide, methanol, hydrogen cyanide, water, mercaptans, hydrogen sulfide, ammonia and arsine. The hydrogenated, absorbed, compressed hydrocarbon stream in the hydrogenation line 224 may be cooled to between 20 and 50° C. and fed to a first adsorption unit 230 to remove these impurities.

The hydrogenated, absorbed, compressed hydrocarbon stream in the hydrogenation line 224 may be delivered to a first adsorption unit 230 for adsorbing at least one of water, mercury, ammonia and mercaptan, one of methanol and hydrogen cyanide, one of carbon dioxide, carbonyl sulfide, hydrogen sulfide and preferably all of these from the hydrogenated, absorbed, compressed hydrocarbon stream to provide a first adsorbed hydrogenated, absorbed, compressed hydrocarbon stream in a first adsorbed line 248 comprising ethylene.

The first adsorption unit 230 may include a first adsorbent for removing water in a first adsorbent bed 233. The first adsorbent may be UOP 3A-EPG, a 1/16 inch potassium exchanged type A molecular sieve adsorbent having the formula: Kₓ[(AlO₂)ₓ(SiO₂)_{y}].zH₂O. The first adsorption unit may also include a second adsorbent for removing mercury in a second adsorbent bed 234. The second adsorbent may be HgSIV-3 1/16 inch, a silver loaded type A molecular sieve specially formulated to adsorb mercury having the formula: Mₓ[(AlO₂)ₓ(SiO₂)_{y}].aAg₂O.zH₂O [M=Na, K]. The first adsorption unit may also include a third adsorbent for adsorbing polar molecules, water, mercaptans, methanol, ammonia, hydrogen cyanide, carbonyl sulfide, hydrogen sulfide, carbon dioxide in a third adsorbent bed 235. The third adsorbent may be UOP AZ-300 comprising a special alumina zeolite composite 7×14 beads with low reactivity. The first adsorption unit may also include a fourth adsorbent for adsorbing residual carbonyl sulfide and hydrogen sulfide in a fourth adsorbent bed 236. The fourth adsorbent may be SG-731 available from UOP LLC, comprising a spherical specialty alumina adsorbent. The first adsorption unit 230 will reduce concentrations of water, mercury, ammonia, methanol, mercaptans, hydrogen sulfide, carbon dioxide, carbonyl sulfide and hydrogen cyanide into acceptable ranges. Each of the first through fourth adsorbents may be provided in one to four separate vessels or stacked in fewer than four vessels. In an aspect, a single adsorption vessel may contain all first through fourth adsorbent beds 233-236 stacked in the same order from top to bottom. Flow is preferably down flow in the first adsorption unit 230.

The first adsorption unit 230 may include an adsorption vessel 232 including beds 233-236 of the first, second, third and fourth adsorbents just mentioned that adsorb water, mercury, ammonia, methanol, mercaptans, hydrogen sulfide, carbon dioxide, carbonyl sulfide and hydrogen cyanide by contact with the hydrogenated, absorbed, compressed hydrocarbon stream to provide a first adsorbed stream in a first adsorbed line 236. In an aspect, the first adsorption unit 230 may include a first adsorption vessel 232 with first adsorbent beds 237 comprising adsorbent beds 233-236 and a second adsorption vessel 238 with second adsorbent beds 240 comprising adsorbent beds 243-246 each with beds of the first, second, third and fourth adsorbent, respectively, that adsorb water, mercury, ammonia, methanol, mercaptans, hydrogen sulfide, carbon dioxide, carbonyl sulfide and hydrogen cyanide by contact with the hydrogenated, absorbed, compressed hydrocarbon stream to provide a first adsorbed, hydrogenated, absorbed, compressed hydrocarbon stream in the first adsorbed line 248.

In one aspect, the first adsorption vessel 232 and the second adsorption vessel 238 may operate in swing bed mode. In an embodiment, valving is arranged such that the first adsorbent beds 237 in the first adsorption vessel 232 receives the hydrogenated, absorbed, compressed hydrocarbon stream in the hydrogenated hydrocarbon line 224 to adsorb impure materials while the second adsorbent beds 240 in the second adsorption vessel 238 are out of communication with the hydrogenation line 224. The second adsorbent beds 240 may undergo regeneration with a desorption gas such as nitrogen gas from regeneration line 242 to remove adsorbed materials from the second adsorbent beds 240 while out of communication with the hydrogenation line 224. When the first adsorbent beds 237 are spent, valving may be rearranged such that the second adsorbent beds 240 in the second adsorption vessel 238 receives the hydrogenated, absorbed, compressed hydrocarbon stream in hydrogenated hydrocarbon line 224 to adsorb impure materials while the first adsorbent beds 237 in the first adsorption vessel 232 are out of communication with the hydrogenation line 224. The first adsorbent beds 237 may undergo regeneration with the desorption gas from the regeneration line 242 to remove adsorbed materials from the first adsorbent beds 237 while out of communication with the hydrogenation line 224. Regeneration gas laden with impurities exits the first adsorption unit 230 in regeneration exhaust line 244. The regeneration exhaust line 244 may be treated to capture mercury present therein and avoid its release to the atmosphere.

The first adsorbed, hydrogenated, absorbed, compressed hydrocarbon stream adsorbed stream in the first adsorbed line 248 still may contain arsine concentration over an acceptable level. For example, the arsine level may be between 100 and 500 wppb, but it may only be acceptable at a concentration below 15 wppb. Hence, the first adsorbed, hydrogenated, absorbed, compressed hydrocarbon stream in the first adsorbed line 248 may be fed to a second adsorption vessel 250 containing a fifth adsorbent in a fifth adsorbent bed 252. The first adsorbed stream is contacted with a fifth adsorbent in the fifth adsorbent bed 252 to adsorb arsines and provide a second adsorbed hydrogenated, absorbed, compressed hydrocarbon stream in a second adsorbed line 254. The fifth adsorbent may be a lead oxide alumina silicate. The fifth adsorbent may be AR-201 adsorbent available from Unicat Catalyst Technologies, Inc. in Alvin, Tex. The second adsorbed stream in the second adsorbed line 254 may comprise no more than about 15 wppb of arsine. The second adsorbed stream in the second adsorbed line 254 may have a pressure of about 1.5 to about 2 MPa.

In an aspect, now that impurity concentrations have been removed to acceptable levels, ethylene may be recovered from the second adsorbed, hydrogenated, absorbed, compressed hydrocarbon stream in the second adsorbed line 254. Ethylene may be recovered in a cold box or in a C₂ splitter column 260. For example, a C₂ splitter column 260 may be in downstream communication with the second adsorbent vessel 250 and be configured to recover ethylene from the second adsorbed, hydrogenated, absorbed, compressed hydrocarbon stream in the second adsorbed line 254. The C₂ splitter column 260 may recover a high purity ethylene product stream such as greater than 99.5 mol % in a net splitter overhead line 262 and a C₂+ bottoms stream in a splitter bottoms line 264. The C₂+ stream in the splitter bottoms line 264 may be delivered to a steam cracking unit as steam cracking feed or be subjected to further recovery. The C₂ splitter column 260 may operate at about 3.5 to about 4 MPa and an overhead temperature of about −30° C. (−22° F.) to about −50° C. (−58° F.).

Accordingly, a waste dry gas stream can be utilized to produce a valuable high purity ethylene stream.

Any of the above lines, units, separators, columns, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring components, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

### Specific Embodiments

While the following is described in conjunction with specific embodiments, it will be understood that this description is intended to illustrate and not limit the scope of the preceding description and the appended claims.

A first embodiment of the invention is a process for removing light gases from a hydrocarbon stream including ethylene, ethane and heavier hydrocarbons, the process comprising (a) absorbing carbon dioxide, hydrogen sulfide and carbonyl sulfide from the hydrocarbon stream by contact with a solvent to provide an absorbed hydrocarbon stream; (b) selectively hydrogenating acetylene in the absorbed hydrocarbon stream to ethylene to provide a hydrogenated hydrocarbon stream; (c) adsorbing at least one of water, mercury, ammonia and mercaptan and one of methanol and hydrogen cyanide and arsine from the hydrogenated hydrocarbon stream to provide an adsorbed hydrocarbon stream comprising ethylene. An embodiment of the invention is one, any or all of prior embodiments in this paragraph up through the first embodiment in this paragraph further comprising washing the absorbed hydrocarbon stream with water to absorb amines from the absorbed hydrocarbon stream. An embodiment of the invention is one, any or all of prior embodiments in this paragraph up through the first embodiment in this paragraph further comprising converting nitrous oxides to ammonia and oxygen to water while selectively hydrogenating acetylene in the absorbed hydrocarbon stream. An embodiment of the invention is one, any or all of prior embodiments in this paragraph up through the first embodiment in this paragraph further comprising adsorbing at least one of water, mercury, ammonia and mercaptan and one of methanol and hydrogen cyanide arsine in a first adsorption unit and adsorbing arsine in a second adsorption unit. An embodiment of the invention is one, any or all of prior embodiments in this paragraph up through the first embodiment in this paragraph further comprising regenerating adsorbent in the first adsorption unit with a desorption gas. An embodiment of the invention is one, any or all of prior embodiments in this paragraph up through the first embodiment in this paragraph further comprising compressing the hydrocarbon stream before the absorption step. An embodiment of the invention is one, any or all of prior embodiments in this paragraph up through the first embodiment in this paragraph further comprising scrubbing acid gases from the hydrocarbon stream prior to compressing the hydrocarbon stream. An embodiment of the invention is one, any or all of prior embodiments in this paragraph up through the first embodiment in this paragraph further comprising recovering ethylene from the adsorbed hydrocarbon stream. An embodiment of the invention is one, any or all of prior embodiments in this paragraph up through the first embodiment in this paragraph further comprising converting acetylene to ethylene, oxygen to water and nitrous oxides to ammonia with hydrogen previously resident in the absorbed hydrocarbon stream. An embodiment of the invention is one, any or all of prior embodiments in this paragraph up through the first embodiment in this paragraph further comprising sensing at least one parameter of the process; generating a signal or data from the sensing; and transmitting the signal or the data.

A second embodiment of the invention is a process for removing light gases from a hydrocarbon stream including ethylene, ethane and heavier hydrocarbons, the process comprising compressing the hydrocarbon stream to provide a compressed hydrocarbon stream; absorbing carbon dioxide, hydrogen sulfide and carbonyl sulfide from the compressed hydrocarbon stream by contact with a solvent to provide an absorbed hydrocarbon stream; (b) selectively hydrogenating acetylene in the absorbed hydrocarbon stream to ethylene to provide a hydrogenated hydrocarbon stream; (c) adsorbing at least one of water, mercury, ammonia and mercaptan and one of methanol and hydrogen cyanide and arsine from the hydrogenated hydrocarbon stream to provide an adsorbed hydrocarbon stream comprising ethylene. An embodiment of the invention is one, any or all of prior embodiments in this paragraph up through the second embodiment in this paragraph further comprising washing the absorbed hydrocarbon stream with water to absorb amines from the absorbed hydrocarbon stream. An embodiment of the invention is one, any or all of prior embodiments in this paragraph up through the second embodiment in this paragraph further comprising converting nitrous oxides to ammonia and oxygen to water while selectively hydrogenating acetylene in the absorbed hydrocarbon stream. An embodiment of the invention is one, any or all of prior embodiments in this paragraph up through the second embodiment in this paragraph further comprising adsorbing at least one of water, mercury, ammonia and mercaptan and one of methanol and hydrogen cyanide arsine in a first adsorption unit and adsorbing arsine in a second adsorption unit. An embodiment of the invention is one, any or all of prior embodiments in this paragraph up through the second embodiment in this paragraph further comprising regenerating adsorbent in the first adsorption unit with a desorption gas. An embodiment of the invention is one, any or all of prior embodiments in this paragraph up through the second embodiment in this paragraph further comprising recovering ethylene from the adsorbed hydrocarbon stream. An embodiment of the invention is one, any or all of prior embodiments in this paragraph up through the second embodiment in this paragraph further comprising scrubbing acid gases from the hydrocarbon stream prior to compressing the hydrocarbon stream. An embodiment of the invention is one, any or all of prior embodiments in this paragraph up through the second embodiment in this paragraph further comprising converting acetylene to ethylene, oxygen to water and nitrous oxides to ammonia with hydrogen previously resident in the absorbed hydrocarbon stream.

A third embodiment of the invention is a process for removing light gases from a hydrocarbon stream including ethylene, ethane and heavier hydrocarbons, the process comprising (a) absorbing carbon dioxide, hydrogen sulfide and carbonyl sulfide from the hydrocarbon stream by contact with a solvent to provide an absorbed hydrocarbon stream; (b) selectively hydrogenating acetylene in the absorbed hydrocarbon stream to ethylene, nitrous oxides to ammonia and oxygen to water to provide a hydrogenated hydrocarbon stream; (c) adsorbing at least one of water, mercury, ammonia and mercaptan and one of methanol and hydrogen cyanide and arsine from the hydrogenated hydrocarbon stream to provide an adsorbed hydrocarbon stream comprising ethylene. An embodiment of the invention is one, any or all of prior embodiments in this paragraph up through the third embodiment in this paragraph further comprising adsorbing at least one of water, mercury, ammonia and mercaptan and one of methanol and hydrogen cyanide arsine in a first adsorption unit and adsorbing arsine in a second adsorption unit.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for removing light gases from a hydrocarbon stream including ethylene, ethane and heavier hydrocarbons, the process comprising:
(a) absorbing carbon dioxide, hydrogen sulfide and carbonyl sulfide from the hydrocarbon stream by contact with a solvent to provide an absorbed hydrocarbon stream;
(b) selectively hydrogenating acetylene in the absorbed hydrocarbon stream to ethylene to provide a hydrogenated hydrocarbon stream; and
(c) adsorbing at least one of water, mercury, ammonia and mercaptan and one of methanol and hydrogen cyanide and arsine from the hydrogenated hydrocarbon stream to provide an adsorbed hydrocarbon stream comprising ethylene.

2. The process of claim 1 further comprising washing the absorbed hydrocarbon stream with water to absorb amines from the absorbed hydrocarbon stream.

3. The process of claim 2 further comprising converting nitrous oxides to ammonia and oxygen to water while selectively hydrogenating acetylene in the absorbed hydrocarbon stream.

4. The process of claim 1 further comprising adsorbing at least one of water, mercury, ammonia and mercaptan and one of methanol and hydrogen cyanide arsine in a first adsorption unit and adsorbing arsine in a second adsorption unit.

5. The process of claim 4 further comprising regenerating adsorbent in said first adsorption unit with a desorption gas.

6. The process of claim 1 further comprising compressing said hydrocarbon stream before said absorption step.

7. The process of claim 1 further comprising scrubbing acid gases from said hydrocarbon stream prior to compressing said hydrocarbon stream.

8. The process of claim 1 further comprising recovering ethylene from said adsorbed hydrocarbon stream.

9. The process of claim 1 further comprising converting acetylene to ethylene, oxygen to water and nitrous oxides to ammonia with hydrogen previously resident in said absorbed hydrocarbon stream.

10. The process of claim 1 further comprising:
sensing at least one parameter of the process;
generating a signal or data from the sensing; and
transmitting said signal or said data.

11. A process for removing light gases from a hydrocarbon stream including ethylene, ethane and heavier hydrocarbons, the process comprising:
(a) compressing said hydrocarbon stream to provide a compressed hydrocarbon stream;
(b) absorbing carbon dioxide, hydrogen sulfide and carbonyl sulfide from the compressed hydrocarbon stream by contact with a solvent to provide an absorbed hydrocarbon stream;
(c) selectively hydrogenating acetylene in the absorbed hydrocarbon stream to ethylene to provide a hydrogenated hydrocarbon stream; and
(d) adsorbing at least one of water, mercury, ammonia and mercaptan and one of methanol and hydrogen cyanide and arsine from the hydrogenated hydrocarbon stream to provide an adsorbed hydrocarbon stream comprising ethylene.

12. The process of claim 11 further comprising washing the absorbed hydrocarbon stream with water to absorb amines from the absorbed hydrocarbon stream.

13. The process of claim 11 further comprising converting nitrous oxides to ammonia and oxygen to water while selectively hydrogenating acetylene in the absorbed hydrocarbon stream.

14. The process of claim 11 further comprising adsorbing at least one of water, mercury, ammonia and mercaptan and one of methanol and hydrogen cyanide arsine in a first adsorption unit and adsorbing arsine in a second adsorption unit.

15. The process of claim 14 further comprising regenerating adsorbent in said first adsorption unit with a desorption gas.

16. The process of claim 11 further comprising recovering ethylene from said adsorbed hydrocarbon stream.

17. The process of claim 11 further comprising scrubbing acid gases from said hydrocarbon stream prior to compressing said hydrocarbon stream.

18. The process of claim 11 further comprising converting acetylene to ethylene, oxygen to water and nitrous oxides to ammonia with hydrogen previously resident in said absorbed hydrocarbon stream.

19. A process for removing light gases from a hydrocarbon stream including ethylene, ethane and heavier hydrocarbons, the process comprising:
(a) absorbing carbon dioxide, hydrogen sulfide and carbonyl sulfide from the hydrocarbon stream by contact with a solvent to provide an absorbed hydrocarbon stream;
(b) selectively hydrogenating acetylene in the absorbed hydrocarbon stream to ethylene, nitrous oxides to ammonia and oxygen to water to provide a hydrogenated hydrocarbon stream; and
(c) adsorbing at least one of water, mercury, ammonia and mercaptan and one of methanol and hydrogen cyanide and arsine from the hydrogenated hydrocarbon stream to provide an adsorbed hydrocarbon stream comprising ethylene.

20. The process of claim 19 further comprising adsorbing at least one of water, mercury, ammonia and mercaptan and one of methanol and hydrogen cyanide arsine in a first adsorption unit and adsorbing arsine in a second adsorption unit.
